(19) United States
(12) Patent Application Publication
(10) Pub. No.: US 2009/0175835 A1
(43) Pub. Date: Jul. 9, 2009

(54) COMPOSITION FOR TREATING DAMAGE OF CENTRAL OR PERIPHERAL NERVE SYSTEM

(75) Inventors: Young-Sook Son, Seoul (KR); Guang-Fan Chi, Seoul (KR); Mi-Ra Kim, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: KOREA INSTITUTE OF RADIOLOGICAL & MEDICAL SCIENCES, Seoul (KR)

(21) Appl. No.: 12/278,722

(22) PCT Filed: Jan. 19, 2007
(86) PCT No.: PCT/KR07/00316

§ 371 Date: Oct. 30, 2008

(30) Foreign Application Priority Data
Feb. 7, 2006 (KR) 10-2006-0011849

(51) Int. Cl. A61K 35/30; C12N 5/08; A61P 25/16; A61P 25/00

(52) U.S. Cl. 424/93.7; 435/377

(57) ABSTRACT

The present invention provides a composition for treating damage of central or peripheral nerve system comprising neural precursor cell derived from subcutaneous tissue; neuron obtained by differentiating the neural precursor cell; or oligodendrocyte or Schwann cell obtained by differentiating the neural precursor cell; a use of neural precursor cell derived from subcutaneous tissue, neuron obtained by differentiating the neural precursor cell, or oligodendrocyte or Schwann cell obtained by differentiating the neural precursor cell, for the manufacture of an agent for treating damage of central or peripheral nerve system; a method for treating damage of central or peripheral nerve system which comprises administrating to a mammal a therapeutically effective amount of neural precursor cell derived from subcutaneous tissue, neurons obtained by differentiating the neural precursor cell, or oligodendrocyte or Schwann cell obtained by differentiating the neural precursor cell. Further, the present invention provides a method of preparing the neural precursor cell, neurons, oligodendrocyte or Schwann cell of the present invention.

### TECHNICAL FIELD

The present invention provides a composition for treating damage of central or peripheral nerve system comprising neural precursor cell derived from subcutaneous tissue, neuron obtained by differentiating the neural precursor cell, or oligodendrocyte or schwann cell obtained by differentiating the neural precursor cell; a use of neural precursor cell derived from subcutaneous tissue, neuron obtained by differentiating the neural precursor cell, or oligodendrocyte or schwann cell obtained by differentiating the neural precursor cell for the manufacture of an agent for treating damage of central or peripheral nerve system; a method for treating damage of central or peripheral nerve system which comprises administrating to a mammal a therapeutically effective amount of neural precursor cell derived from subcutaneous tissue, neurons obtained by differentiating the neural precursor cell, or oligodendrocyte or schwann cell obtained by differentiating the neural precursor cell. Further, the present invention provides a method of preparing the neural precursor cell, neuron, oligodendrocyte or schwann cell of the present invention.

### BACKGROUND ART

Recently, a new method has been tried to treat neural diseases by differentiating embryonic stem cell or neural stem cell to neuron cell, and transplanting the neuron into brain. This method has shown good effects such as improvement of the clinical symptoms and survival of the transplanted cells in animal models suffering from Parkinson disease and stroke. Thus, it is expected that transplantation of neuron cell derived from stem cell into brain can be applied to patients suffering from Parkinson disease, stroke amyotrophic lateral sclerosis or spinal cord injury in the near future.

Neural stem cell can be defined as undifferentiated cell having persistent proliferation ability, i.e., self renewalability, and multipotent differentiation ability that can be differentiated into neuron, astrocyte, oligodendrocyte, etc. Neural stem cell is differentiated to neuron or glial cell through neural or glial precursor cell stage. Thus, the mechanism research of inducing differentiation to neuron or glial cell or inhibiting such differentiation is very important in disease pathogenesis of neural system and therapeutic strategy as well as development of neural system.

Some questions have been presented on which parts neural stem cell exists in brain, and when those cells originate and disappear in neural system development stage. In the researches to solve these questions, Nestin, an intermediate filament, is used as specific marker to neural stem cell or neural precursor cell. It is known that Nestin positive neural stem cell exists in neural plate, before neural tube is formed, i.e., an early stage of spinal cord development. After the formation of neural tube, Nestin positive neural stem cell exists in peri ventricular zone of neural tube. These neural stem cells persistently repeat asymmetrical division producing two types of daughter cells: one is neural stem cell itself, and the other is neuron/glial cell differentiated from neural stem cell.

Recent researches disclosed that neural stem cell known to exist in early nervous system development stage also exists in adult mammal. For the past 100 years, it was an established theory that new neuron cannot be produced in adult mammal. But, recently, it was found that neural stem cell exists in hippocampus and subventricular zone of central nerve system of adult human.

However, hippocampus and subventricular zone are located deep inside of human brain. Thus, it is difficult to utilize these neural stem cells as autogenic source for treating damage of nervous system such as Parkinson disease, stroke, amyotrophic lateral sclerosis, spinal cord injury, motor nerve injury, peripheral nerve traumatic damage, etc.

Thus, many researches have been performed to find new accessible neural stem cell source. As a result, it was reported that mononuclear cell fraction from bone marrow cell, umbilical blood cell, or fetal hepatocyte has differentiation ability to neural system cell.

However, bone marrow cell does not have good accessibility, and umbilical blood cell's preservation is currently limited to some new born babies. Thus, these cells are not suitable for self graft of neural system injury treatment yet. And, embryonic stem cell has ethical problem and many practical difficulties for industrial utilization.

Also, preexistent donor stem cells such as bone marrow cell, embryonic stem cell, etc. have a problem that they should be necessarily proliferated to a large number for clinical applications. For solving such problem, PCT/JP02/11294 disclosed a method for inducing differentiation of immortalized mesodermal stem cell to neural system cell, and suggested that immortalizing mesodermal stem cell is a method for mass proliferation of cell in vitro. But, it is a problem that the cells transfected with immortalized gene may become abnormal cells.

As a trial to overcome these problems, Freda Miller group (Jean G. Toma., Nature Cell Biology, vol 3, September 2001) in Canada reported that they identified multi potent stem cell that can be differentiated to neuron, glial cell, myocyte or adipocyte, from scalp tissue by using spheroid culture for 3 months (U.S. patent application Ser. No. 09/991,480). If skin dermal tissue can be used as donor stem cell, it has advantages that it can be easily obtained for clinical applications, compared with other stem cells.

Freda Miller group's experiment solved some problems in other stem cells, but their method needs 3 months of cell culture, and so is difficult to utilize in practice. Also, various cell modifications such as transformation, dedifferentiation, etc. may occur during the long period of culture.

Accordingly, the present inventors developed a method for inducing adult human's skin dermal tissue to neural precursor cells within a short period of 6 to 12 days (Korean Patent Application No. 2004-0099394).

### DISCLOSURE OF INVENTION

### Technical Problem

In a following research for improving the former research of isolating the neural precursor cell from skin dermal tissue as a source of neural stem cell, the present inventors discovered that neural precursor cell to form sphere and express nestin can be induced from a cell isolated from subcutaneous tissue cell of newly born and adult rat.

Skin dermal tissue may comprise a lot of appendages originated from epidermal cell such as sebaceous gland, sweat gland, hair follicles, etc. So, in case the neural precursor cell forming sphere can be isolated from dermal tissue, and can be prepared as cell therapy agent, there is a problem that different cells originated from appendages of skin are mixed with the neural precursor cell, and so the rate of sphere-forming cell is lower than skin subcutaneous tissue.

On the other hand, in case of extracting subcutaneous tissue from skin underneath, the skin dermal tissue can be obtained without damage of adnexa of skin. And, subcutaneous tissue is broadly distributed over human body, and contains mainly fat tissue and connecting tissue, and so has relatively simple anatomy structure and good accessibility. Thus, subcutaneous tissue has merits that a sufficient amount thereof can be extracted, with preserving epidermis and dermal layer of skin and minimizing scar area on skin surface, and can be used for self neural treatment agent development. Therefore, if neural precursor cell can be induced from subcutaneous tissue and can be used as the cell therapeutic agent, this agent would be very practical and useful for clinical application.

Accordingly, one object of the present invention is to provide a composition for treating damage of central or peripheral nerve system comprising neural precursor cell derived from subcutaneous tissue, neuron obtained by differentiating the neural precursor cell, or oligodendrocyte or schwann cell obtained by differentiating the neural precursor cell; a use of neural precursor cell derived from subcutaneous tissue, neuron obtained by differentiating the neural precursor cell, or oligodendrocyte or schwann cell obtained by differentiating the neural precursor cell for the manufacture of an agent for treating damage of central or peripheral nerve system; a method for treating damage of central or peripheral nerve system which comprises administrating to a mammal a therapeutically effective amount of neural precursor cell derived from subcutaneous tissue, neuron obtained by differentiating the neural precursor cell, or oligodendrocyte or schwann cell obtained by differentiating the neural precursor cell; and a method of preparing the neural precursor cell, neuron, oligodendrocyte or schwann cell of the present invention.

### Technical Solution

The present invention provides a composition for treating damage of central or peripheral nerve system comprising neural precursor cell derived from subcutaneous tissue as an active ingredient.

In one embodiment of the present invention, the neural precursor cell derived from subcutaneous tissues is cell to form neurosphere and express nestin and/or SOX10.

In other embodiment of the present invention, the neural precursor cell is cell obtained from sphere which is formed by culturing subcutaneous tissue cell in a medium comprising N2 supplement, bFGF and EGF.

Also, the present invention provides a composition for treating damage of central or peripheral nerve system comprising neuron which is obtained by differentiating neural precursor cell derived from subcutaneous tissue as an active ingredient.

In an example of the present invention, the neuron is cell which is obtained by differentiating neural precursor cell derived from subcutaneous tissue in a neurobasal medium comprising NT3.

The present invention also provides a composition for treating damage of central or peripheral nerve system comprising oligodendrocyte or schwann cell which is obtained by differentiating neural precursor cell derived from subcutaneous tissue.

In an example of the present invention, the oligodendrocyte or schwann cell is cell which is obtained by culturing neural precursor cell derived from subcutaneous tissue in a medium comprising retinoic acid, and differentiating them in a medium comprising serum, forskolin, bFGF, PDGF and heregulin.

Moreover, the present invention provides a use of neural precursor cell derived from subcutaneous tissue, neuron obtained by differentiating the neural precursor cell, or oligodendrocyte or schwann cell obtained by differentiating the neural precursor cell for manufacture of an agent for treating damage of central or peripheral nerve system.

Further, the present invention provides a method for treating damage of central or peripheral nerve system which comprises administrating to a mammal a therapeutically effective amount of neural precursor cell derived from subcutaneous tissue, neuron obtained by differentiating the neural precursor cell, or oligodendrocyte or schwann cell obtained by differentiating the neural precursor cell.

In the present invention, the damage of central or peripheral nerve system includes Parkinson's disease, stroke, amyotrophic lateral sclerosis, spinal cord injury, motor nerve injury or peripheral nerve traumatic damage.

Still further, the present invention provides a method of preparing neural precursor cell, neuron, oligodendrocyte or schwann cell from subcutaneous tissue cell

The present method prepares neural precursor cell from subcutaneous tissue cell which comprises culturing the subcutaneous tissue cell in a medium comprising N2 supplement, bFGF and EGF.

Still further, the present invention provides a method for differentiating the neural precursor cell derived from subcutaneous tissue cell according to the method of culturing neuron in a neurobasal medium comprising NT3.

Still further, the present invention provides a method for differentiating the neural precursor cell derived from subcutaneous tissue cell according to the method of culturing oligodendrocyte or schwann cell in a medium comprising retinoic acid and then in a medium comprising serum, forskolin, bFGF, PDGF and heregulin.

Below, the present invention will be described in detail.

In the present invention, the neural precursor cell induced from subcutaneous tissue are obtained by isolating subcutaneous tissue cell from skin subcutaneous tissue, and then culturing it in medium comprising N2 supplement, bFGF and EGF.

In order to obtain neural precursor cell induced from subcutaneous tissue in a large amount, it is efficient to follow the process of isolating subcutaneous tissue cell from skin subcutaneous tissue; selecting spheroid forming cell; performing monolayer culture of the spheroid forming cell up to passage 5, to obtain a large amount of cells; and inducing the expanded cells to spheres.

For example, i) subcutaneous tissue cell is cultured in a medium comprising N2 supplement, bFGF and EGF to form sphere, and ii) the sphere is cultured in a medium comprising B27 supplement, bFGF and EGF.

Or, subcutaneous tissue cell is cultured in DMEM/F12 medium comprising N2 supplement; the sphere forming cell is monolayer cultured in a medium comprising serum; and then the expanded cell is cultured in a medium comprising N2 supplement, bFGF and EGF or a medium comprising G5 supplement.

The neural precursor cell derived from subcutaneous tissue may be differentiated to neuron by being cultured in a neurobasal medium comprising NT3, or be differentiated to oligodendrocyte or schwann cell by being cultured in a medium comprising retinoic acid, followed by being cultured in a medium comprising serum, forskolin, bFGF, PDGF, and heregulin.

The method of isolating, culturing and differentiating neural precursor cell according to the present invention may be explained by newly born rat in detail below.

(1) Isolated cell from new born rat's subcutaneous tissue is suspended and cultured in DMEM/F12 medium comprising N2 supplement (Step 1).

(2) The detached cell in Step 1 is harvested through centrifugation, then plated, and cultured in DMEM/F12 medium comprising B27 supplement to reform spheres (Step 2).

(3) The spheres formed in Step 2 are broken into individual cells, which are subcultured in DMEM/F12 medium comprising B27 supplement to form spheres (Step 3).

(4) The spheres subcultured in Step 3 are cultured in a neurobasal medium comprising NT3 for 8-10 days to induce differentiation to neuron cells (Step 4).

(5) The differentiation to neuron cells is confirmed by immunofluorescence analysis with neuron specific markers and special gene expression analysis with RT-PCR of mRNA (Step 5).

Also, the method of isolating, culturing and differentiating neural precursor cells according to the present invention may be explained in mature rat as follows.

(1) The isolated cell from mature rat's subcutaneous tissue is suspended and cultured in DMEM/F12 medium comprising N2 supplement (Step 1).

(2) The detached cell in Step 1 is harvested through centrifugation, then plated in DMEM/F12 medium comprising serum, and monolayer-cultured up to passage 5 (Step 2).

(3) The monolayer cultured P5 cell in Step 2 is cultured in a medium comprising N2 supplement, bFGF and EGF on coated dish for 3 days, then all the medium is changed to DMEM/F12 medium comprising G5 supplement, and the cell is cultured for 6 days to form spheres (Step 3).

(4) The cell in Step 3 is cultured in alpha-MEM medium comprising FBS and retinoic acid for 4 days (Step 4).

(5) The cell in Step 4 is subcultured to P4-P5 in a medium of inducing differentiation of sphere forming cell to schwann cell phenotype, comprising alpha-MEM medium, FBS, forskolin, bFGF, PDGF-AA, heregulin1-beta1 and NT3 (Step 5).

(6) The cell in Step 5 is isolated and plated to coated dish, then cultured for 3 days in the medium of inducing differentiation of sphere forming cell to schwann cell phenotype of Step 5. To identify whether the cell of Step 5 is differentiated to schwann cell, immunofluorescence analysis is conducted and gene expression analysis is conducted by using RT-PCR. Also, other cells in Step 5 are labeled with PKH26 fluorescence dye in vitro, then microinjected to the rat's spinal cord injury area, and analyzed to identify their function in 8 weeks from the injection (Step 6).

As a result, it was found that the subcutaneous tissue cell can be differentiated to neural precursor cell, neuron, oligodendrocyte or schwann cell. Also, it was confirmed that the schwann cell which is obtained by differentiating neural precursor cell derived from subcutaneous tissue plays an important role in recovering a rat from spinal cord injury through in vivo experiments.

Accordingly, the present invention provides a therapeutic agent for damage of central or peripheral nerve system comprising neural precursor cell derived from subcutaneous tissue, neuron obtained by differentiating the neural precursor cell, or oligodendrocyte or schwann cell obtained by differentiating the neural precursor cell, and a use of neural precursor cell derived from subcutaneous tissue, neurons obtained by differentiating the neural precursor cell, or oligodendrocyte or schwann cell obtained by differentiating the neural precursor cell for the manufacture of an agent for treating damage of central or peripheral nerve system.

The damage of central or peripheral nerve system by using the present therapeutic agent may include Parkinson's disease, stroke, amyotrophic lateral sclerosis, spinal cord injury, motor nerve injury or peripheral nerve traumatic damage.

In the present invention, the components of the medium which is required to culture subcutaneous tissue cell to neural precursor cell or differentiate the neural precursor cell to neuron may be replaced by other components showing equal effects, if necessary. The components for culture of neural precursor cell or differentiation of neural precursor cell into neuron are well known to a skilled artisan who can select appropriate components, if necessary.

Also, it is obvious that the contents of the components used for the culture medium of neural precursor cell or the differentiation medium into neuron may be varied according to the object or level that a skilled artisan intends to culture or differentiate.

Only, in the present examples, bFGF can be used, preferably in the range of 1-100 ng/ml, more preferably in the range of 10-40 ng/ml. And, heparin can be used preferably in the range of 0.5-20 μg/ml, more preferable in the range of 2-10 μg/ml, but is not limited thereto.

The constitution of N2 supplement used in the present invention is insulin 500 μg/ml+human transferrin 10 mg/ml+progesterone 0.63 μg/ml+putresin 1.611 mg/ml+selenites 0.52 μg/ml. The constitution of G5 supplement is insulin μg/ml+human transferrin 5 mg/ml+selenite 0.52 μg/ml+biotin 1.00 μg/ml+hydrocortison 0.36 μg/ml+FGF 0.50 μg/ml+EGF 1.0 μg/ml. The constitution of B27 supplement is not known to the art, but B27 supplement has been marketed and broadly used in the art.

Also, the present invention provides a method for treating damage of central or peripheral nerve system which comprises administrating to a mammal a therapeutically effective amount of neural precursor cell derived from subcutaneous tissue, neuron obtained by differentiating the neural precursor cell, or oligodendrocyte or schwann cell obtained by differentiating the neural precursor cell.

In the present invention, the effective dosage of neural precursor cell derived from subcutaneous tissue, neuron obtained by differentiating the neural precursor cell, or oligodendrocyte or schwann cell obtained by differentiating the neural precursor cell is 1×10⁴ to 1×10⁷ cells/kg. However, the dosage may be modified depending on the weight, age, sex or severity of damage of patient.

The therapeutic agent according to the present invention can be administered into human bodies parentally, locally, for example, by intravenous injection, intra-arterial injection, cerebrospinal fluid injection, etc. For effective administration, the ingredients are suspended or dissolved in a pharmacologically acceptable carrier, preferably, water-soluble carrier such as physiological saline.

The advantages and features of the present invention and the method of revealing them will be explicit from the following examples described in detail. However, it should be distinctly understood that the present invention is not limited thereto, and can be embodied and practiced in many other ways. It is obvious that the following examples are to complete the disclosure of the invention and to indicate the scope of the present invention to a skilled artisan completely. The present invention will be defined only by the scope of the claims.

### Advantageous Effects

The present invention demonstrates that the cell from skin subcutaneous tissue can be induced to neural precursor cell. In case subcutaneous tissue is used as a source of neural precursor cell, it is advantageous in several points: having good accessibility, compared with central nerve system's neural stem cell or embryonic stem cell; providing sufficient area for clinical application; being more economical source than dermal cell; avoiding contamination of skin appendages such as sebaceous gland, sweat gland or hair follicle, and minimizing scar formation in donor after separating subcutaneous tissue. Therefore, the present cell therapy agent comprising neural precursor cell derived from subcutaneous tissue, neuron obtained by differentiating the neural precursor cell, or oligodendrocyte or schwann cell obtained by differentiating the neural precursor cell is very useful for treating damage of central nervous system (CNS) and peripheral nervous system (PNS).

### DESCRIPTION OF THE DRAWINGS

FIG. 1 is a picture of Haematoxyline and Eosin-staining of the section of rat skin, showing the subcutaneous tissue beneath the dermal tissue.

FIG. 2 shows spheres formed by culturing the cells isolated from subcutaneous tissue in DMEM/F12 (Dulbecco's modified eagle medium: nutrient mixture F-12) medium comprising N2 supplement, bFGF (basic fibroblast growth factor) and EGF (epidermal growth factor) for 3 days.

FIG. 3 shows spheres grown by 17 days of continuous culturing.

FIG. 4 shows neural spheres obtained by culturing the cells isolated from new born rat's hippocampus in a neurobasal medium comprising B27, bFGF and EGF for 17 days.

FIG. 5 shows morphologic changes of the cells spread out from spheres originated from subcutaneous tissue, after culturing the spheres in DMEM/F12 medium comprising FBS (fetal bovine serum) for 24 hours.

FIG. 6 shows the morphologic changes of the cells spread out from spheres originated from hippocampus, after culturing the spheres in DMEM/F12 medium comprising FBS (fetal bovine serum) for 24 hours.

FIG. 7 shows the bipolar morphology of the cells changed in 24 hours after they are induced to neuron cells in a neurobasal medium comprising NT3 (neurotrophin 3).

FIGS. 8a to 8d show the morphology of the cells at 8^{th} day after the cells of FIG. 7 are induced to neuron.

FIG. 9 shows the morphology of the cells at 10^{th} day after the cells of FIG. 7 are induced to neuron.

FIG. 10 shows the results of immunofluorescence analysis for the cells of FIG. 8 by using neural specific markers such as nestin, GFAP (Glial fibrillary acidic protein), βIII-tubulin, neurofilament 200 KD, oligodendrocytes, CSPG (chondroitin sulfate proteoglycan), A2B5, etc.

FIG. 11 shows the results of RT-PCR (reverse transcriptase-polymerase chain reaction) analysis for the cells before and after (at 8^{th} day) the spheres derived from subcutaneous tissue are induced to differentiate to neuron.

FIG. 12 shows a sphere-like structure formed when the spheres originated from mature rat subcutaneous tissue were cultured in a medium containing N2 and bFGF for 7 days.

FIG. 13 shows the morphology of the cells that spheroid-forming cells isolated from subcutaneous tissue of mature rat are sub-cultured to Passage 5 in DMEM/F12 containing FBS.

FIG. 14 shows the state of the cells sub-cultured to passage 5, beginning to form clusters when cultured in DMEM/F12 medium containing G5 supplement for 24 hours.

FIG. 15 shows the spheres re-formed when the monolayer subcultured cells (p 5) were cultured in DMEM/F12 medium comprising G5 supplement for 4 days

FIG. 16 shows the morphologic changes of the spheres when the spheres formed from p5 cells were cultured in alpha-MEM (minimum essential medium alpha medium) comprising FBS and all-trans-retinoic acid (RA) for 4 hours.

FIGS. 17 and 18 show the cells' morphology when the cells of FIG. 16 were isolated in alpha-MEM medium comprising FBS and retinoic acid for 4 days, then cultured in alpha-MEM medium comprising FBS, forskolin, bFGF, PDGF-AA (platelet-derived growth factor AA) and heregulin1-β1 to induce differentiation to schwann cells for 2 days.

FIG. 19 shows a net-like morphology of the cells when the cells were cultured in alpha-MEM medium comprising FBS, forskolin, bFGF, PDGF-AA and heregulin 1-β1 on PDL-coated dish for 6 days, to induce differentiation to schwann cells.

FIG. 20 shows the morphology of schwann cells isolated from sciatic nerve of new born rat after primary culture for 2 days as control culture

FIG. 21 shows the results of immunofluorescence analysis to schwann cell's specific markers of O4, A2B5, GFAP, P75, S100 and RIP after the schwann cell induced from mature rat's subcutaneous cells was subcultured in α-MEM medium comprising 10% FBS, 5 μM forskolin, 10 ng/ml bFGF, 5 ng/ml PDGF-aa, 200 ng/ml heregulin 1-β1, 10 ng/ml NT3.

FIG. 22 shows the results of RT-PCR analysis on the schwann cell's markers for the cell derived from the mature rat's subcutaneous tissue at different time points.

FIG. 23 shows the results of immunofluorescence analysis for 5HT, GABA and CGRP antigens expression in the injured central area of the spinal cord injury (SCI) test group.

FIG. 24 shows the results of immunofluorescence analysis for GFAP and CSPG antigens expression in the injured central area of the SCI test group.

FIG. 25 shows the results of immunofluorescence analysis for P75 and neurofilament 200 KD antigens expression in the injured central area of the SCI test group.

FIG. 26 shows the results of immunofluorescence analysis for MBP and P0 antigens expression in the injured central area of the SCI test group.

FIG. 27 shows the results of immunofluorescence analysis for OMG antigen expression in the injured central area of the SCI test group.

FIG. 28 shows the results of immunofluorescence analysis for fibronectin and neurofilament 200 KD antigens expression in the injured central area of the SCI test group.

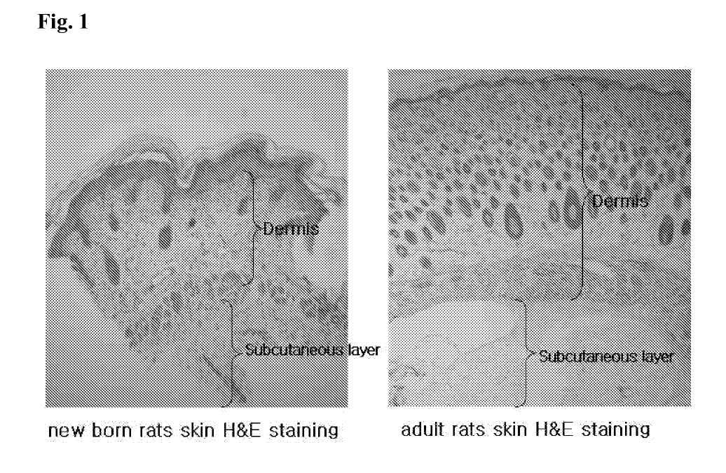

### EXAMPLE 1

### Confirmation of Differentiation Induction from Skin Subcutaneous Tissue Cell of New Born Rat to Neuron

1-(1). Isolation of Skin Subcutaneous Tissue Cell and Spheroid Culture

The skin including subcutaneous tissue was dissociated from back of new born rat. Under sterile condition, the subcutaneous tissue was dissected from the skin's dermal and epidermal tissue under anatomic microscope. The separated subcutaneous tissue was washed with same volume of PBS (phosphate-buffered saline) to remove red blood cell and debris. Then, the tissue was digested with 0.1% collagenase for 40 min at 37° C., and the enzyme activity was neutralized by DMEM containing 10% FBS (fetal bovine serum) and centrifuged at 1500 rpm/min for 5 minutes. The pellet was resuspended and digested in PBS containing 0.1% DNase for 1 minute. After that, the suspension was mechanically separated by 10 times of pipetting in DMEM, and filtered through 40 μm cell strainer. Then, the suspension was washed and centrifuged 2 times at 1500 rpm/min for 5 minutes.

The cells in pellet were evenly suspended in DMEM/F12 (3:1) medium comprising 20 ng/ml bFGF (R&D), 20 ng/ [symbol] EGF (R&D), N2 supplement, 2 μg/ [symbol] heparin (Sigma), 1% penicillin/streptomycin (JBI), and plated in 100 mm dish.

After 3 days, non-attached cells were collected by centrifugation at 1500 rpm/min for 5 minutes. These cells were suspended in DMEM/F12(3:1) medium comprising 1% B27 supplement (Gibco), 20 ng/ml bFGF, 20 ng/ [symbol] EGF, 2 μg/ [symbol] heparin, 1% penicillin/streptomycin, and plated in 100 mm dish. ⅔ of the medium was refreshed every 3 days, and the spheres formed therefrom were dissociated with a fire-polished pasteur pipette every 7-10 days.

1-(2). Confirmation of Formation of the Sphere of Cell Derived from Skin Subcutaneous Tissue

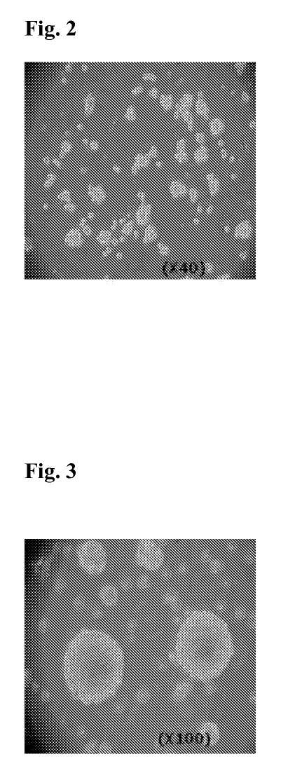

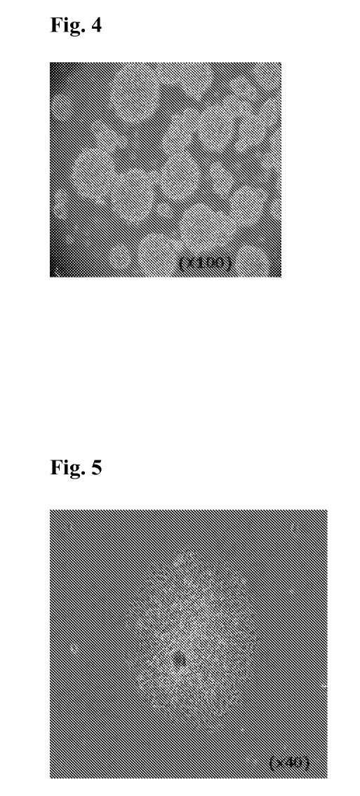

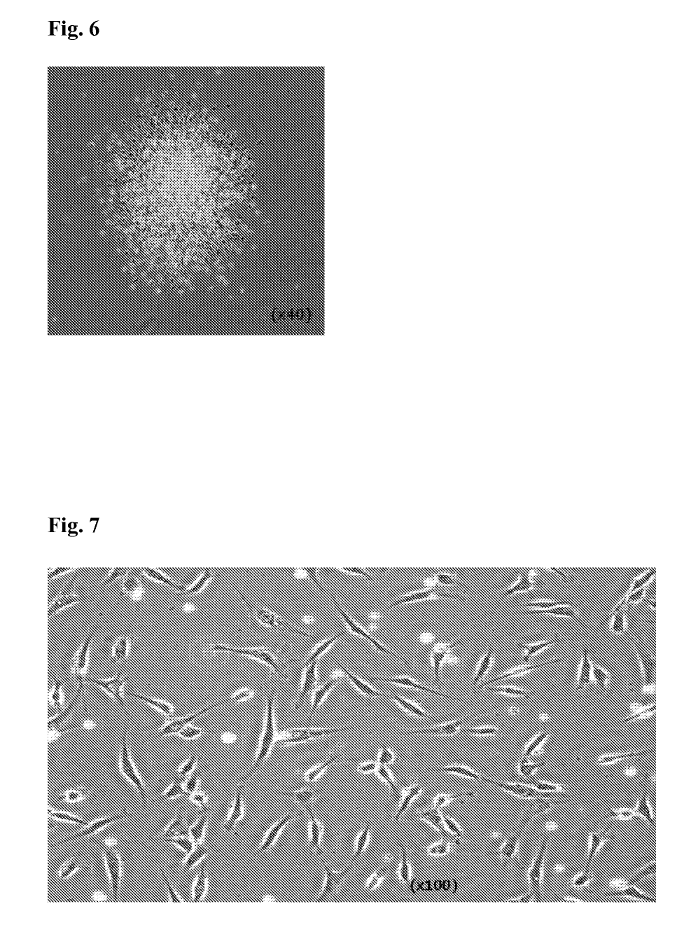

According to the section 1-(1) of Example 1, the sphere was formed from the cell derived from subcutaneous tissue. FIG. 2 shows the sphere-like structure formed from the isolated cell from the skin subcutaneous tissue (at 3^{rd} day). In case that the sphere-like structure was cultured continuously, the neurosphere-like morphology was formed, as shown in FIG. 3 (at 17^{th} day). It can be confirmed that the morphology is similar to the neurosphere of hippocampus of new born rat (at 17^{th} day) of FIG. 4.

FIG. 5 shows the morphologic change after the neurosphere-like structure derived from subcutaneous tissue was cultured in DMEM/F12 (3:1) containing 10% FBS for 24 hours. It can be known that the morphology is very similar to one that neurosphere from hippocampus was cultured in DMEM/F12 (3:1) containing 10% FBS for 24 hours.

2-(1). Induction of Differentiation to Neuron Cell

The sphere was dissociated to single cell with fire-polished pasteur pipette, and the suspension of cell was diluted to 70,000 cells/ml of density in neurobasal medium (Gibco) comprising 1 μg/ [symbol] laminin (Sigma), N2 supplement (Invitrogen), 20 ng/ [symbol] bFGF (R&D) and 20 ng/ [symbol] EGF (R&D). 500 [symbol] (or 10 ml) of the suspension was plated on the PDL(poly-D-lysin)/laminin coated flask and cover slips in 24 well. After 48 hours, the medium was discarded and replaced to differentiation medium comprising neurobasal medium (Gibco), 1 μg/ [symbol] laminin (sigma), N2 supplement (Gibco) and 20 ng/ [symbol] NT3(R&D). ⅔ volume of the medium was changed every 2 days and constantly cultured.

2-(2). Confirmation of Differentiation Induction to Neuron Cell

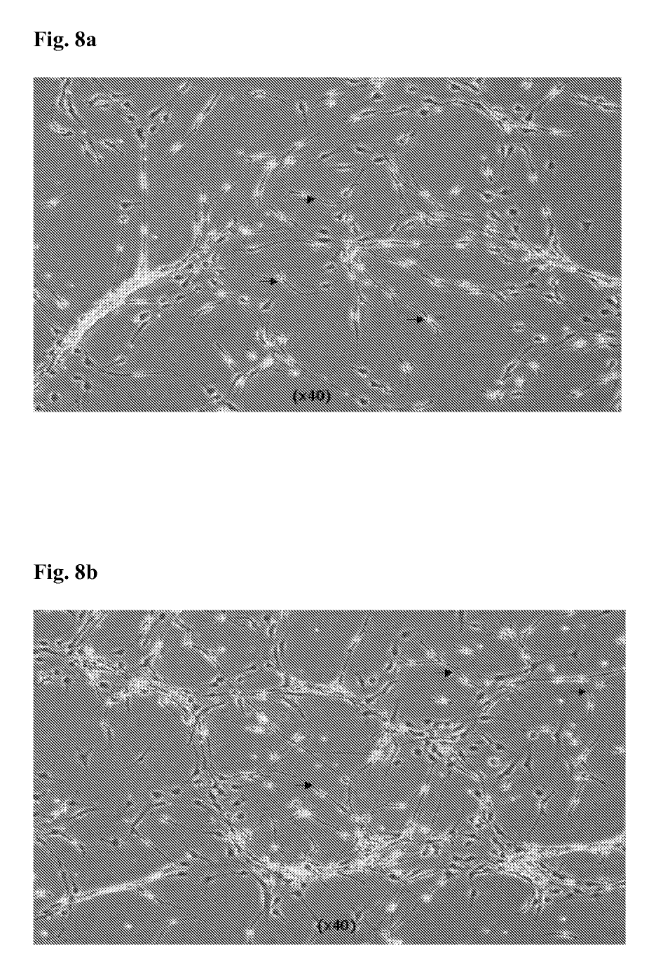

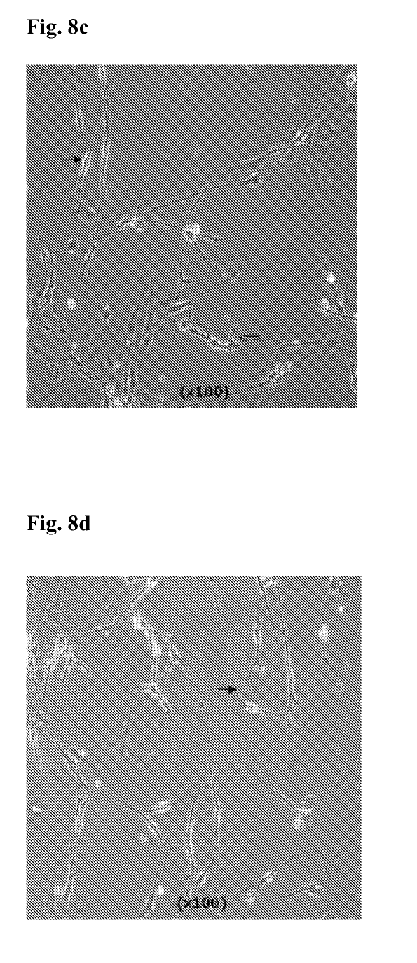

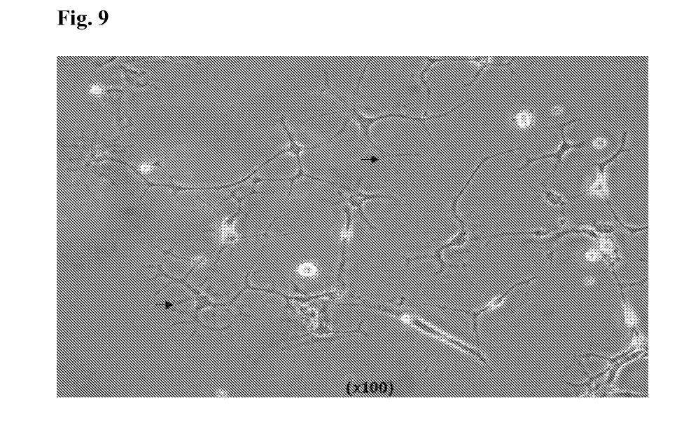

The skin subcutaneous tissue cell became the morphology of neuron cell after it was induced to differentiation to neuron cell according to the section 2-(1) of Example 1. FIG. 7, FIGS. 8a-8d, and FIG. 9 show the cell's morphology after different time points, 24 hours, 8 days, and 10 days.

FIG. 7 shows the elongated morphology of the cells after they were induced to differentiation to neuron for 24 hours. FIGS. 8a-8d show that most of the cells have neuron-like morphology after 8 days when the differentiation to neuron was induced. In FIG. 8, the black arrows indicate the neuron cell's morphology, and the white arrows indicate oligodendrocyte's morphology. FIG. 9 shows that the cells exhibit neuron cell's morphology or oligodendrocyte's morphology (black arrow) after 10 days when the differentiation to neuron was induced.

3-(1). Confirmation of Differentiation by Immunofluorescence Analysis

After culturing the cells for 8 days according to the method 2-(1) of Example 1, the cells in 24-well were fixed in 4% cold formaldehyde in PBS for 30 minutes before performing immunofluorescence analysis.

The cells fixed on the cover slips were washed 3 times, each for 10 minutes, permeabilizated in 0.2% X-100 triton for 10 minutes, and washed 3 times in PBS, each for 10 minutes. Then, these cover slips were blocked in 10% normal horse serum for 1 hour at room temperature, and then incubated overnight with first antibodies diluted in 5% normal horse serum at 4° C. The primary antibodies used therein were: mouse anti-tubulin-βIII (1:50, Chemicon), mouse anti-nestin (1:50, Chemicon), mouse anti-GFAP (1:100, Chemicon), mouse anti-neurofilament 200 KD (1:100, Chemicon), mouse anti-oligodendrocyte (1:100, Chemicon), mouse anti-CSPG (1:100, Chemicon), mouse anti-A2B5 (1:50, Chemicon). After the primary antibodies were removed, the cover slips were washed in PBS 3 times, each for 10 minutes. These cover slips were incubated with fluorescein-conjugated horse antibody against mouse Ig(H+L) as second antibody diluted in 5% normal horse serum at room temperature for 1.5 hours. Finally, the cover slips were washed 3 times, each for 10 minutes, and the cell's nucleus was counterstained with DAPI. The cover slips were maintained on glass slide and visualized under Leica microscope.

3-(2). Immunofluorescence Analysis Results

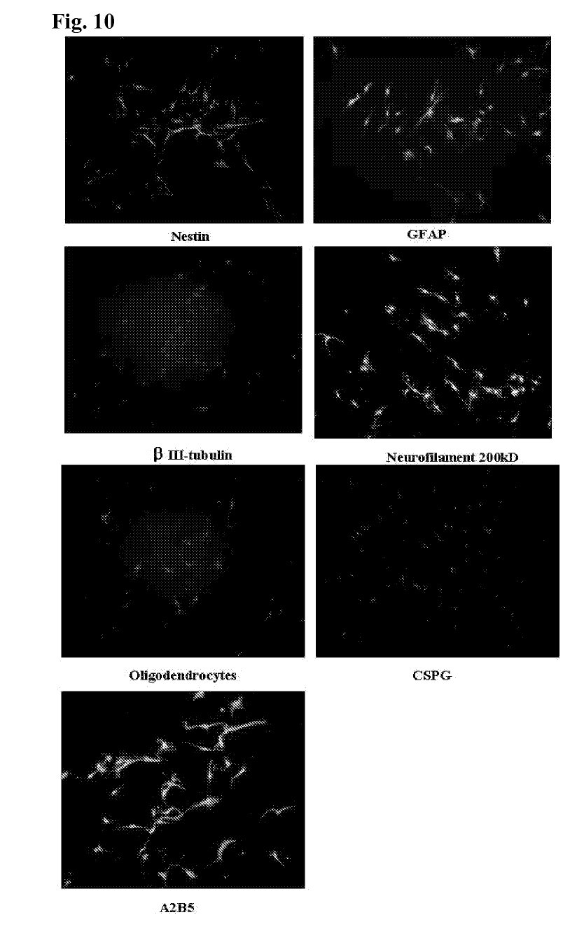

FIG. 10 shows the results of immunofluorescence analysis for nestin, tubulin-βIII, neurofilament 200 KD, Oligodendrocytes, CSPG and A2B5 to neuron cells 8 days after the differentiation was induced. The results are shown in FIG. 10. As shown in FIG. 10, it is confirmed that the cells of subcutaneous tissue shows the properties of neural precursor cell when the differentiation is induced.

The results are summarized in the following table.

| | Level of | |
|---|---|---|
| Name | expression | mark |
| Nestin | ++ | neural stem cell |
| GFAP | ++ | astrocyte |
| Tubulin-β III | + | neuron |
| Neurofilament | ++ | neuron axon |
| 200KD | | |
| CSPG | + | astrocyte |
| Oligodendrocyte | + | oligodendrocyte |
| A285 | ++ | oligodendrocyte |

4-(1). Confirmation of Differentiation by RT-PCR Analysis

After 8 days from the cell culture according to the section 2-(1) of Example 1, the cells were separated from 100 mm culture dish by using EDTA/Trypsin (Gibco). Then, these cells were washed and centrifuged 3 times in PBS, and the total mRNA was extracted by RNeasy mini kit (Qiagen).

According to the instruction of Omniscript RT kit (Qiagen), cDNA was synthesized, and gene expressions were analyzed by PCR as follows.

| Name of | | | Size | |
|---|---|---|---|---|
| primer | Light primer sequence (5′-3′) | Right primer sequence (5′-3′) | (bp) | A. T(° c.) |
| Nestin | ACTGCAGAAGAGGACCTGGA | ACTTCCCACTCCTGTGGTTG | 393 | 56 |
| Tubulin-β III | ATTCCCTGGTCAGCTCAATG | AACAGGCCGTAAACTGCTC | 444 | 56 |
| GFAP | GGTGGAGAGGGACAATCTCA | TGTGAGGTCTGCAAACTTGG | 382 | 58 |
| SOX10 | AAGCTCTGGAGGTTGCTGAA | GGTCCAGCTCAGTCACATCA | 478 | 60 |
| GAPDH | ATGGGAAGCTGGTCATCAAC | GGATGCAGGGATGATGTTCT | 440 | 58 |

4-(2). RT-PCR Analysis Results

Nestin, tubulin-βIII, GFAP, Sox 10 and GAPDH gene expressions analyses were conducted for the pre-induction cells [the cells of 1-(1)] and the post-induction cells [the cells of 2-(1) 8 days after the differentiation to neuron was induced]. Sox10 is a neural crest stem cell's marker.

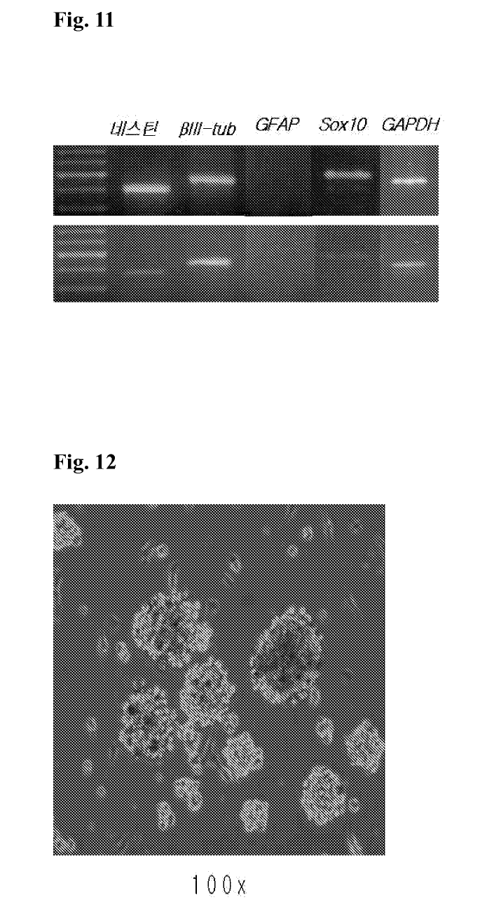

The analysis results are shown in FIG. 11.

After the differentiation to neuron was induced, the expression levels of nestin, tubulin-III and Sox10 were decreased. Assuming that the levels of GAPDH expression are same before and after the differentiation, the expression levels of nestin and/or Sox 10 were decreased at the post-induction, and tubulin-III expression was increased at the post induction.

These results demonstrate that subcutaneous tissue cell has the properties of neural precursor cell, and can be differentiated to neuron under neuron cell-inducing condition.

### EXAMPLE 2

### Confirmation of Differentiation Induction of Subcutaneous Tissue Cell of Mature Rat to Schwann Cell

1-(1). Isolation of Subcutaneous Tissue Cell and Culture of Sphere

Adult female Sprague-Dawley rat (350 g) was anaesthetized, and subcutaneous tissue was carefully dissected from the skin dermal beneath under aseptic condition. This tissue was washed with PBS (comprising 100 U/ml penicillin and 100 ug/ml streptomycin) to remove the contaminated debris and red blood cell. Then, the tissue was mechanically minced to about 1 mm³ by using a surgical scissor, treated with 0.5% collagenase type I, and stood for 1 hour in 37° C. Then, the tissue was treated with DMEM/F12 (1:1) comprising 10% FBS, the same volume as the collagenase, to inactivate the collagenase, and centrifuged in 1500 rpm/min for 5 minutes. The supernatant was carefully discarded, and the remainder was washed with DMEM/F12 (3:1) for 2 times, and then filtered through 40 μm strainer filter to remove debris.

The filtrate was centrifuged to isolate subcutaneous tissue cells, which were suspended in DMEM/F12 (3:1) medium comprising N2 supplement, 20 ng/ml EGF, 20 ng/ml bFGF and 2 ug/ml heparin, and plated in 75 cm² flask. The medium was refreshed every 3 days.

After 7˜10 days, the sphere formed therein was isolated by centrifuging from culture medium, and dissociated to single cells by using fire-polished Pasteur pipette. Such dissociated cells were spun down and resuspended in DMEM/F12 (3:1) comprising 10% FBS, and plated in 75 cm² flask at 5×10⁵/ml density. Two thirds (⅔) of the medium was replaced every 3 days, and the cells were separated to single cells by using 0.5% EDTA/trypsin when the cells' confluence reached 90%. These cells were diluted at the ratio of 1:3, replated to 75 cm² flask, and then subcultured to Passage 5.

1-(2). Confirmation of the Formation of Sphere of the Cell Derived from Subcutaneous Tissue

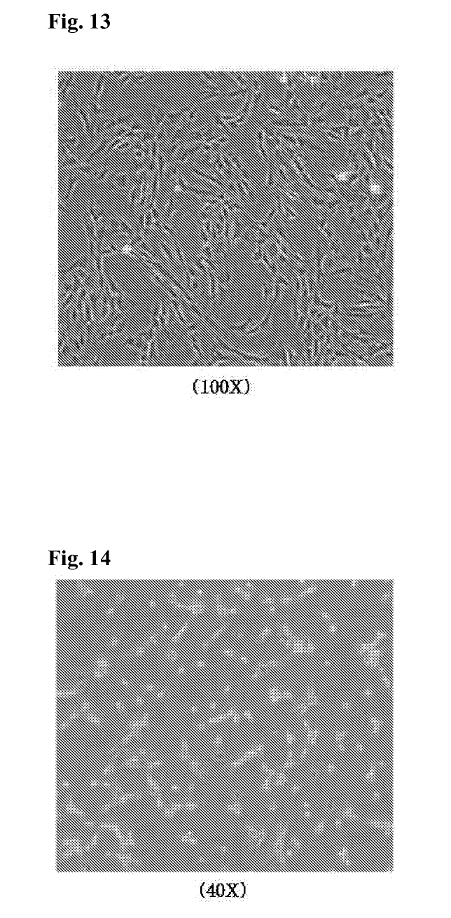

According to the section 1-(1) of Example 2, the sphere was formed by culturing the cell derived from subcutaneous tissue cell. FIG. 12 shows a sphere-like structure formed by culturing the isolated subcutaneous tissue cell from mature rat in a medium comprising N2 supplement and bFGF for 7 days. In case that the subcultured cell at passage 5 (the cell as shown in FIG. 13) was cultured in DMEM/F12 (3:1) comprising 20 ng/ml bFGF (R&D), 20 ng EGF (R&D), N2 supplement (Gibco), 2 μg/ml heparin and 1% penicillin/streptomycin (replaced ⅔ of medium every 3 days) consecutively for 6 days, or the subcultured cell was cultured in the above medium for 3 days and in a neurobasal medium comprising G5 supplement, 2 μg/ml laminin and 2 μg/ml heparin for 24 hours, the cell's morphology was as shown in FIG. 14. Also, in case that the cell was cultured in the above medium (G5 containing medium) for 3-4 days, it was observed that the sphere was formed as shown in FIG. 15.

2. Differentiation Induction to Schwann Cells and Confirmation thereof.

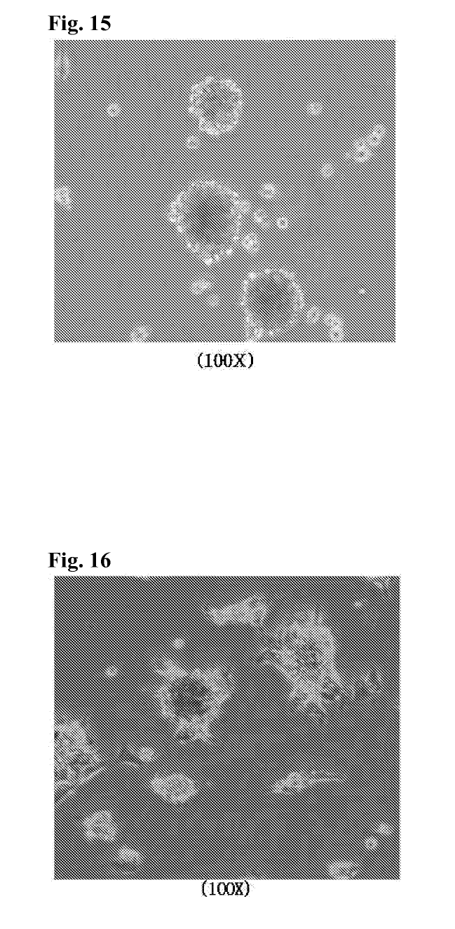

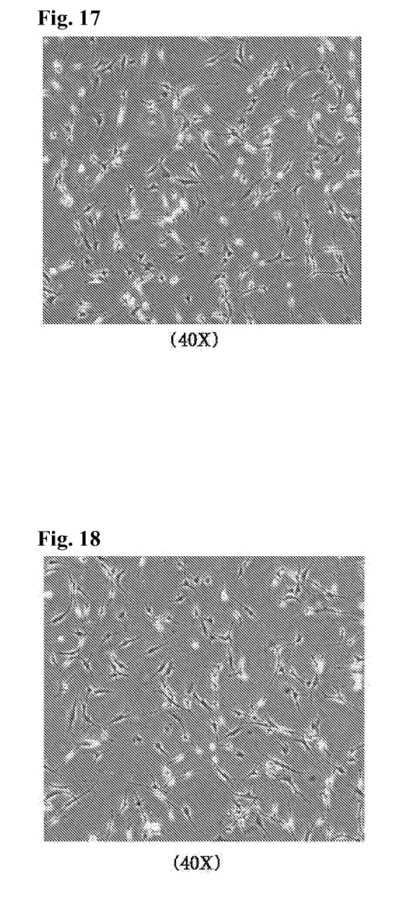

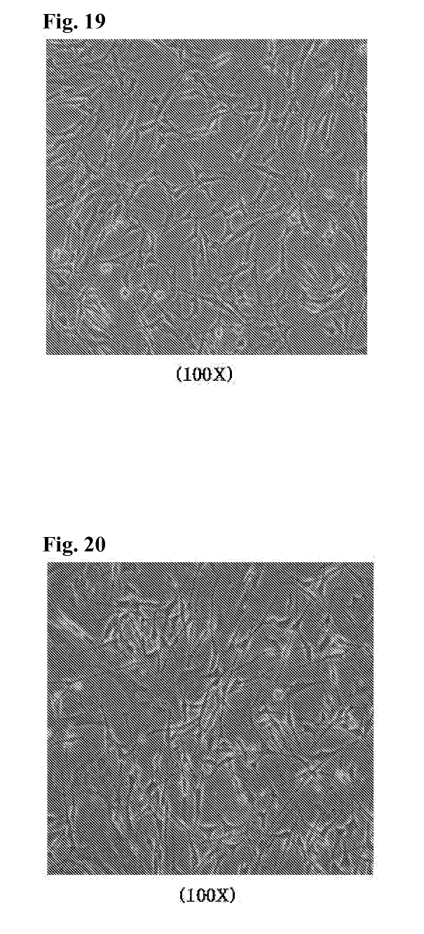

The sphere of the section 1-(2) after 7 days was separated by centrifugation, then plated to poly-L-lysine coated dish, and cultured in α-MEM medium comprising 10% FBS, 35 ng/ml all-trans retinoic acid, 100 u/ml penicillin and 100 ug/ml streptomycin for 4 days. FIG. 16 shows the morphologic change of the sphere after 4 days' culture. After the 4 days of culture, the cell was separated by using trypsin-EDTA solution, then plated to poly-L-lysine coated dish, and cultured in α-MEM medium comprising 10% FBS, 5 μB forskolin, 10 ng/ml bFGF, 5 ng/ml PDGF-aa, 200 ng/ml heregulin1-beta1 for 8 days. ⅔ of the medium was refreshed every 4 days. FIGS. 17-18 show the cell's morphology after 2 days, and FIG. 19 shows the cell's morphology after 6 days. It could be confirmed that the cell's morphology was similar to the morphology of primarily cultured Schwann cell for 2 days from new born rat. After confluence, the cell was separated and subcultured for 20 days in the same medium except replacing forskolin with 10 ng/ml NT3 (neurotrophin-3).

3-(1) Confirmation of Differentiation by Immunofluorescence Analysis

The cell induced to Schwann cell was trans-differentiated, sub-cultured, seeded on cover slips, and cultured for 4 days in α-MEM medium comprising 10% FBS, 5 μM forskolin 10 ng/ml bFGF, 5 ng/ml PDGF-aa, 200 ng/ml heregulin-β1 and 10 ng/ml NT3, and immunofluorescence analysis was performed for the cell. The cell was fixed with a solution of 4% paraformaldehyde (PFA) in PBS for 30 min. After washed with PBS 3 times each for 10 min, the cell was permeabilized in 0.2% Triton X-100 in PBS, and washed with PBS 3 times each for 10 min. Then, non-specific antigens were blocked with 20% goat serum in PBS for 1 hour, and incubated overnight with the primary antibodies: mouse anti-O4 (1:500; Chemicon), mouse anti-A2B5 (1:500; Chemicon), mouse anti-RIP (1:1000; Chemicon), goat anti-PDGFra (1:50; Santa Cruz), mouse anti-P75 (1:200; Chemicon), mouse anti-GFAP (1:200; Chemicon), mouse anti-S100 (1:100; Chemicon) and mouse anti-vimentin (1:200; Chemicon). Then, the cell was washed with PBS 3 times, and incubated with anti-mouse IgG (1:200; Chemicon) as the secondary antibody at room temperature for 1 hour. The results were examined by Leica fluorescence microscope.

The markers for cell and tissue immuno staining analysis were shown in the following table.

| | |
|---|---|
| O4 | immature schwann cell and oligodendrocyte |
| A2B5 | Schwann cell and Oligodendrocyte |
| RIP | Schwann cell and oligodendrocyte |
| S-100 protein | Schwann cell |
| P75 | Schwann cell |
| GFAP | Schwann cell and astrocyte |
| CSPG (chondroitin sulfate | inhibitor of axon regeneration |
| proteoglycan) | |
| Neurofilament 200 KD | Neuron axon |
| MBP (myelin basic protein) | major constituent of CNS, PNS myelin |
| P0 | major constituent of PNS myelin |
| OMG (oligodendrocyte | Expressed by neuron and oligodendrocytes. |
| myelin glycoprotein) | That inhibits neurite outgrowth |
| 5HT | Serotonin neuron |
| GABA | GABA-ergic neuron |
| CGRP (calcitonin gene | Sensory neuron |
| related peptide) | |
| Fibronectin | Fibroblast or other cells |

3-(2). Immunofluorescence Analysis Results

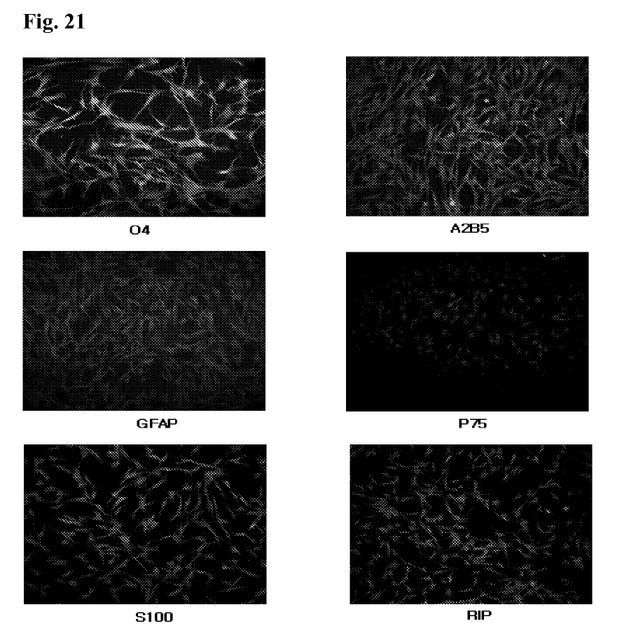

FIG. 21 shows the results of immunofluorescence analysis for schwann cell specific markers after the subcutaneous tissue cell of mature rat was induced to schwann cell phenotype and expanded in α-MEM medium comprising 10% FBS, 5 μM forskolin, 10 ng/ml bFGF, 5 ng/ml PDGF-aa, 200 ng/ml heregulin-beta1 and NT3.

After the differentiation was induced, more than 95% of the cell expressed O4, A2B5, GFAP, S100 and RIP antigens, all of which are schwann cell specific markers, and a part of the cell expressed P75 antigen.

| Name | Level of Expression |
|---|---|
| O4 | ++ |
| A2B5 | ++ |
| GFAP | ++ |
| P75 | + |
| S100 | ++ |
| RIP | ++ |

4-(1). Confirmation of Differentiation by RT-PCR Analysis

The cell at each culture stage was separated by trypsin-EDTA, washed in PBS, and centrifuged at 1500 rpm/min. mRNA was prepared from samples by using RNase mini kit (Queagen), and cDNA was generated with Superscript™ III first stand synthesis system for PCR, as instructed by manufacturers. PCR was performed with 100 ng cDNA by using Perfect PreMix ver 2.1 (Taq, TaKaRa).

The markers used in RT-PCR analysis are listed in the following table.

| | |
|---|---|
| S100b | Schwann cell |
| P75 | Schwann cell |
| Protein 22 | a protein component of peripheral nerve myelin with |
| (PMP22/egr2) | proposed roles in myelin formation and stability |
| SCIP | related to cAMP levels of neuron and making myelin |
| (OCT6/Tst-1) | on neuron axon |
| PLP (myelin | a major constituent of myelin, mainly in CNS myelin |
| proteoliqid | |
| protein or | |
| lipophilin/ | |
| DM20) | |
| Krox-20 | transcription factor of key importance in myelination |
| L1 | adhesion molecule secreted by schwann cells, to |
| | which injured axons attach and grow and important |
| | for ensheathment and myelination. |
| ErbB2 | respective receptor for the applied growth factor |
| | heregulin-beta1 relate to glia cell differentiation and |
| | axon generation |
| PDGFr-aa | Oligodendrocyte |
| NSE | neural precursor cells |

The primers of RT-PCR are listed in the following table.

| | | | size | A.T. |
|---|---|---|---|---|
| Primer | left primer (5′-3′) | right primer (5′-3′) | (bp) | (° C.) |
| S100β | GAGAGAGGGTGACAAG | GGCCATAAACTCCTGG | 169 | 52 |
| | CACAA | AAGTC | | |
| P75 | TGTGTGAAGAGTGCCCA | TCCACAGAGATGCCAC | 496 | 52 |
| | GAG | TGTC | | |
| Pmp22 | TCCTCATCTGTGAGCGA | ACAGACCAGCAAGGA | 163 | 52 |
| | ATG | TTTGG | | |
| SCIP | ATTCCCCGGGAGAATGG | AGGTGCGAGAAGAGG | 126 | 62 |
| | ACGAAAAGAGGAGAGT | CGCGGAAAGAATAAA | | |
| | CC | GTGAC | | |
| Krox-20 | AGATACCATCCCAGGCT | CTCTCCGGTCATGTCA | 300 | 56 |
| | CAGT | ATGTT | | |
| L1 | TGGAAGTGGAGGAAGG | AAGTGGGCATTGCAGA | 202 | 52 |
| | AGAAT | TGTAG | | |
| ErbB2 | AATGCCAGCCTCTCATT | GACTTCGAAGCTGCAG | 235 | 52 |
| | CCTG | CTCC | | |
| PDGFR- | CTGTAACTGGCAGGCTC | GTTGTCTGCAGTACAA | 331 | 55 |
| α | GGAG | GTTGGCG | | |
| NSE | TGTGGTGGAGCAGGAG | GATGCATCGGGAAGG | 556 | 58 |
| | AAGC | GTCAG | | |
| GAPDH | ATGGGAAGCTGGTCATC | GGATGCAGGGATGATG | 440 | 58 |
| | AAC | TTCT | | |
| PLP/ | | | 421 | 55 |
| DM20 | | | | |
| GFAP | | | 430 | 55 |

* Primers GFAP (RD-149-025) and PLP/DM20 (RDP-152-025) from R&D system

4-(2). RT-PCR Analysis Results

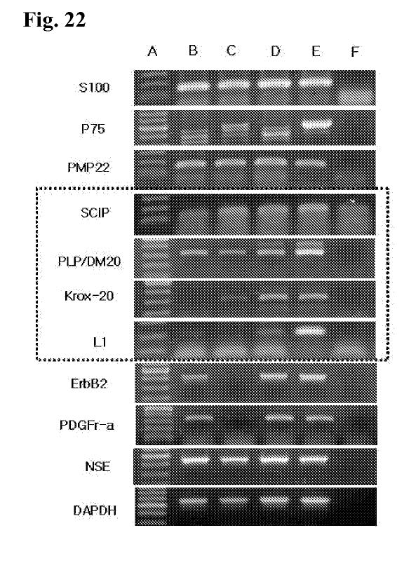

FIG. 22 shows RT-PCR analysis results. The cell derived from subcutaneous tissue of mature rat which is cultured and induced to schwann cell markers according to Example 2, expressed schwann cell markers at different culture time points. Line A represents markers, line B represents gene expression of monolayer culture cell, line C represents gene expression of sphere forming cell, line D represents gene expression after induction to schwann cell phenotype, line E represents positive gene expression of schwann cell from sciatic nerve of rat, and line F represents water as negative control.

After the induction to schwann cell phenotype, the schwann cell specific marker expression (line D) was very similar to normal schwann cell (line E), and the markers of SCIP, PLP, Krox-20 and L1 which have important role in myelin formation, were more expressed than the pre-induction cells.

The immuno cytochemistry and PCR analysis results demonstrated that the cells were differentiated into those having schwann cell properties after the induced differentiation to schwann cell. These cells having schwann cell/oligodendrocyte properties are more easily differentiated to mature oligodendrocyte or schwann cell forming myelin, and are expected to have better treatment effect for CNS or PNS injury.

### EXAMPLE 3

### Cell Transplantation to Spinal Cord Injured Rat

1. Spinal Cord Injury and Cell Transplantation

Adult female Sprague-Dawley rats (N=20) weighing 250-300 g were anaesthetized by intra peritoneal injection of ketamin (80 mg/kg) and rompun (7.4 mg/kg). Laminectomy was carried out at T9-T11 level to expose one spinal cord segment and kept dura intact during the operation. After cleaning the injured area, the spinal cord was squeezed with a clip (20-30 g) perpendicularly on both sides at the same time for 1 minute. Then, the muscle and skin were sutured subsequently with 6-0 sutures. After the operation, these rats were kept on heating pads, observed until fully awake, and then returned to their cages. The rats' bladders were expressed twice a day until reflex urination returned, and cefazolin was administered by intra peritoneal injection every 2 days for 1 weeks.

Three (3) hours before the grafting, P5 cells of the differentiated cells were detached from cell culture flask, resuspended in medium, and then washed 2 times in a medium containing 10% FBS. The cells' membrane was then labeled with PKH26 (Sigma) of fluorescence dye as instructed by the operation manual. Finally, the labeled cells were washed and centrifuged at 1500 rpm/min for 5 minutes, and this procedure was repeated twice. The labeled cells were diluted to the density of 1×10⁵/ul in PBS, and then placed in ice box until transplantation. The cells' viability was confirmed to be more than 95% by trypan blue exclusion after the labeling.

In the testing group, every rat was stereotaxically microinjected into total 5 ul of the labeled cells into the central area of lesion site by using 29-gauge needle attached to a 10 ul Hamilton syringe, 1 ul/min speed in 1.3 mm depth lateral to posterior spinal cord vein for 5 minutes. The needle remained for 5 min and was slowly withdrawn after the injection. Then, the muscle and skin were sutured individually by 6-0 sutures. In the control group, the same volume of PBS was injected to every rat, instead of the cells mixture, and other process was the same as to the testing group. After the operation, these rats kept on heating pads, observed until fully awake, and then returned to their cages. The rats' bladders were expressed twice a day until reflex urination returned, and cefazolin was administered by intra peritoneal injection every 2 days for 1 week. After the surgery, a sample of remaining cells was re-plated overnight to verify viability. All of the rats received immune suppression with a single daily injection of Cyclosporine administered subcutaneously at a dose of 1 mg/100 g starting 3 days before the transplantation, and continued to the end of this animal experiment.

2. Immunohistological Analysis for the Grafting Efficiency of Cell

2-(1). Tissue Preparation

Rats were anesthetized with intra peritoneal injection of ketamin (80 mg/kg) and rompun (7.4 mg/kg), and then opened right atrium. Immediately, 200 ml of PBS containing 1 u/ml of heparin was perfused into left ventricle follow up 300 ml of ice-cold 4% paraformldehyde in 0.1M PBS was perfused. The spinal cord was dissected out, immersed in 4% paraformaldehyde in 0.1M PBS, and kept in 4° C. for 24 hours. Then, these samples were immersed in 30% sucrose for 3 days. The lesion site was identified and horizontality blocked in OCT compound (Tissue Tek, Sakura), and kept at −80° C. until examination.

2-(2). Histology and Immunohistochemisty Analysis

The samples were sectioned longitudinally to include entire lesion area at 20 um thickness by crystal section, and mounted onto Superfrost Plus Slide (VWR international, USA). For immunohistolgical examination, frozen sections were dried completely, and placed in 0.3% Triton X-100 (Sigma, St. Louis, Mo., USA) in 0.01M PBS for 1 hour. To quench endogenous peroxidase activity, the sections were placed in 2% H₂O₂ in 0.01M PBS for 30 minutes. After washing the sections with 0.01 MPBS, non-specific reaction was blocked with 10% goat serum. Then, the section was reacted with the following primary antibodies overnight in 4° C.: rabbit anti serotonin (5HT) (1:500, Chemicon), rabbit anti GABA (1:500, Chemicon), rabbit anti CGRP (1:1000, Chemicon).

Then, the sections were incubated with a biotinylated antibody for 3 hours, and an avidin-biotin peroxidase complex for 1.5 hours (ABC Kit; vector laboratory, Burlingame, Calif., USA) in a humidified chamber. The final reaction for peroxidase was carried out with the DAB preoxidase substrate kit (ABC Kit; vector laboratory, Burlingame, Calif., USA).

For immunofluorescence analysis, the sections were immersed in 0.3% Triton-100 in 0.01M PBS for 1 hour. After washing in 0.01% PBS 3 times, these sections were blocked with 10% normal goat serum or 10% horse serum for 1 hour at room temperature. Then, these sections were applied to the following primary antibodies in 4° C. overnight: mouse anti GFAP (1:200, Chemicon), mouse anti CSPG (1:200, Chemicon), mouse anti P75 (1:500, Chemicon), mouse anti neuralfilament 200 KD (1:200), mouse anti tublin-III beta1(1:300, Chemicon), goat anti MBP (1:50, Santa Cruz), goat anti P0 (1:50, Santa Cruz), goat anti OMG (oligodendrocyte myelin glycoprotein) (1:50, Santa Cruz), rabbit anti fibronectin (1:80, Chemicon), mouse anti ED1(1:100, Serotec).

The following day, these sections were washed 3 times in 0.01M PBS, and then reacted with secondary antibodies (1:200, goat anti mouse, Chemicon or 1:100 goat anti rabbit, Vector) conjugated with FITC or AMCA for 1 hour at room temperature in a humidified chamber. After washing 3 times in 0.01M PBS, these sections were cover-slipped in mounting medium of Vectashield (Vector, Burlingame, Calif.) containing the nuclear counterstain DAPI.

The sections were observed under fluorescence microscope (Leica CTR 4000), and digital images were captured with Zeiss LSM 510 META confocal microscope

2-(3). Results of Histology and Immuno Histochemical Analysis

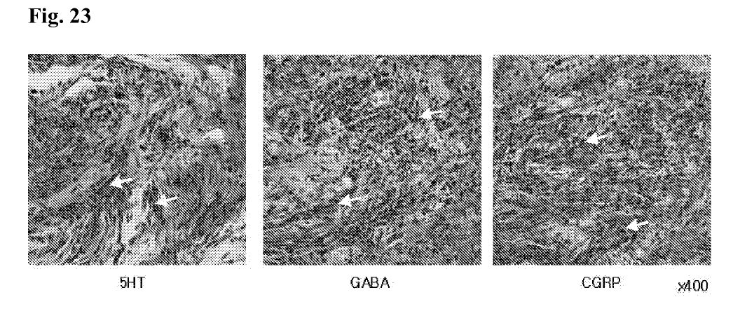

FIG. 23 shows immunohistology photo of serotonin nerve (5HT), GABA-ergic neurons (GABA), and sensory nerve (CGRP) antigen in the injured central area. It shows that many serotonic, GABA-ergic neuron, and some sensory neurons were newly formed in the central side of the injured spinal cord.

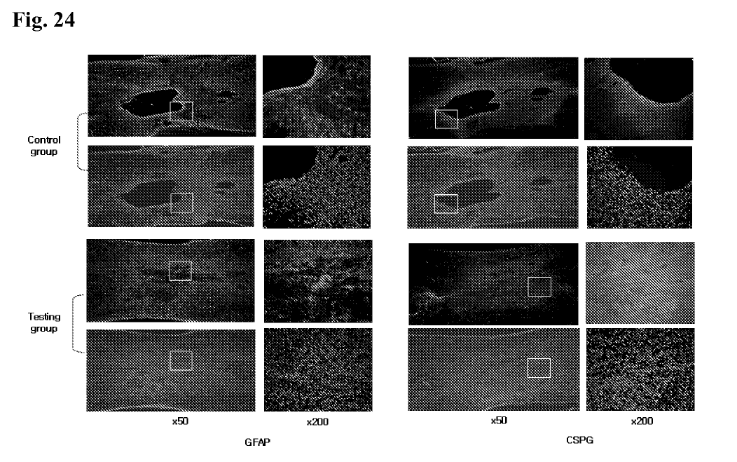

FIG. 24 shows the immunofluorescence appearance of GFAP and CSPG at injured area. The right photo is an enlarged one of the white box in the left photo. GFAP antigen is immature astrocyte marker, and CSPG is inhibitor marker of axon regeneration. It is shown that big cavity was formed in the control groups, but the testing group's cells are distributed evenly in the lesion site. GFAP and CSPG were all expressed in the two groups. However, in the former, both antigens were mostly and strongly expressed at edge of newly formed tissue, and in the latter, evenly expressed in newly formed tissue.

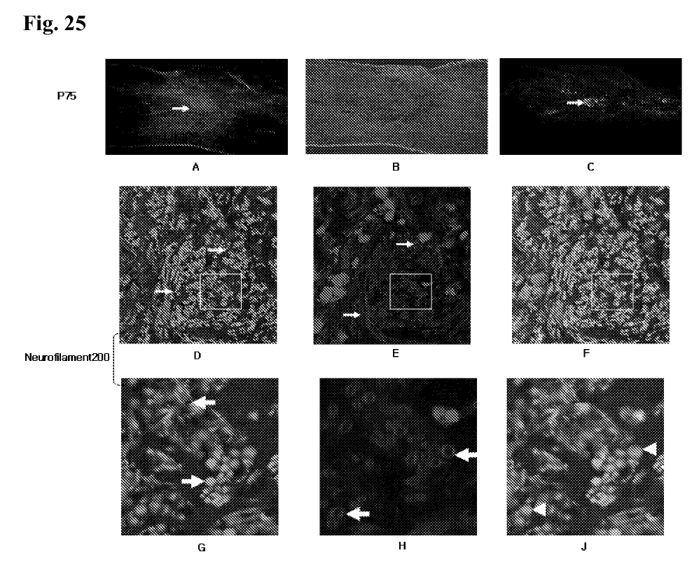

In the immunofluorescence photos of FIGS. 25 to 27, the bright green color represents positive antigen reaction, and the red color represents the sub cultured cells (P5) of the cells that were labeled as PKH26, and induced and differentiated to schwann cell's phenotypes injected into the injured center, in the method 1-(1) of Example 3.

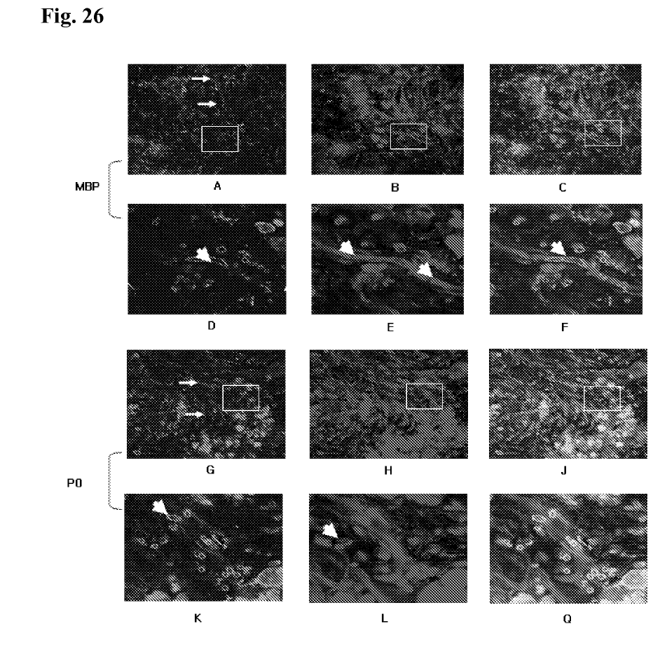

FIG. 25 shows the immunofluorescence photo of P75 and neurofilament 200 KD antigen in the testing group. P75 is the marker to schwann cell, and neurofilament 200 KD is the marker to neuron axon. A of FIG. 26 shows that distinct P75 positive antigen was distributed on all the central area of injured side in the testing group (arrow, A), and B shows the DAPI staining for the cell's nucleus. C of FIG. 25 shows that most of this injured area is filled with red color, transplanted cells. D of FIG. 25 shows a large amount of neurolfilament 200 KD fibers that were spread in this injured area. E of FIG. 25 shows the fibers having distinct red color of PHK26 dye on edge of the cells. F is a merged photo of D and E; and G, H, and J are photos enlarged from the white box in D, E, and F, respectively. In J of FIG. 25, this fiber-like structure with PHK 26 dye has tightly wrapped neurofilament 200 KD positive axons inside (arrowheads).

FIG. 26 shows the immunofluorescence photo to MBP and P0 antigen in the injured central area of spinal cord. The MBP antigen is CNS and PNS myelination markers, and P0 antigen is PNS myelination marker. As shown in FIG. 26, a lot of MBP and P0 antigen positive reactions were observed in the testing group. The myelination proteins wrapped the fibers like tube, and these thickness and distribution were consistent with red color fibers (F, Q), and some fibers has ranvier's node structure (arrow, E). C is a merged photo of A and B, D, E, and F are photos enlarged from white box of A, B and C, respectively. Also, J is a merged photo of G and H, and K, L and Q are photos enlarged from the white box of G, H and J, respectively.

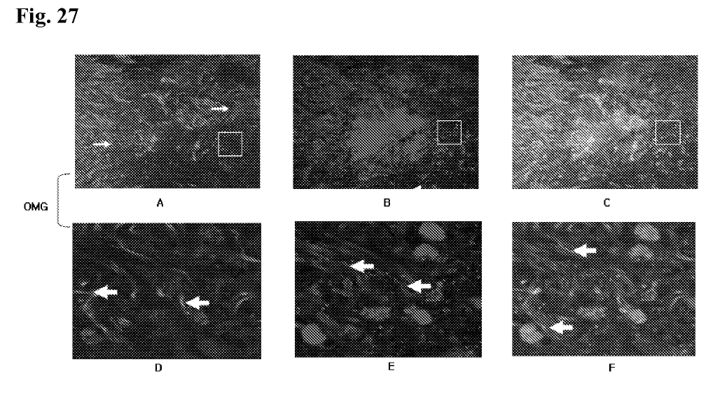

FIG. 27 shows immunofluorescene photo for OMG in the injured area of the testing group. OMG antigen is the marker of neuron and oligodendrocyte. A large number of OMG (A and D) were expressed on edge of the injured area, and contacted with the red fiber surface (arrow, F). C is a merged photo of A and B, D, E and F are photos enlarged from the white box of A, B, and C, respectively.

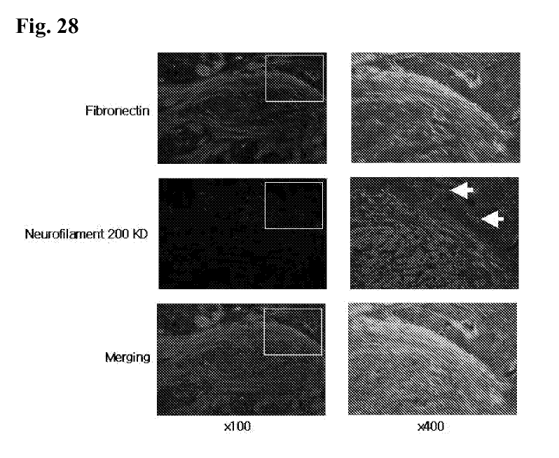

FIG. 28 shows double immunofluorescene staining for fibronectin and neurofilament 200 KD in central area of the injured spinal cord. Fibronectin antigen is a fibroblast marker, and neurofilament 200 KD antigen is neuron axon marker. As known from FIG. 28, a large amount of fibronectin component co-existed with neuron axon in this central site, and some axons were connected to normal spinal cord. In the peripheral area, especially adjacent to canal, a lot of axons were linearly extended to surrounding tissue. The right photo is one to enlarge the white box of the left photo.

1. A composition for treating damage to the central or peripheral nerve system comprising a neural precursor cell derived from subcutaneous tissue as an active ingredient.

2. The composition of claim 1, wherein the neural precursor cell is a cell to form a neurosphere and to express nestin and/or SOX10.

3. The composition of claim 2, wherein the neural precursor cell is a cell obtained from a sphere which is formed by culturing a subcutaneous tissue cell in a medium comprising N2 supplement, bFGF and EGF.

4. A composition for treating damage to the central or peripheral nerve system comprising a neuron which is obtained by differentiating a neural precursor cell derived from a subcutaneous tissue as an active ingredient.

5. The composition of claim 4, wherein the neuron is a cell which is obtained by differentiating the neural precursor cell in a neurobasal medium comprising NT3.

6. A composition for treating damage to the central or peripheral nerve system comprising an oligodendrocyte or schwann cell which is obtained by differentiating a neural precursor cell derived from subcutaneous tissue.

7. The composition of claim 6, wherein the oligodendrocyte or schwann cell is a cell which is obtained by culturing the neural precursor cell in a medium comprising retinoic acid and differentiating the neural precursor cell in a medium comprising serum, forskolin, bFGF, PDGF and heregulin.

8. The composition of claim 1, wherein the damage to the central or peripheral nerve system is Parkinson's disease, stroke, amyotrophic lateral sclerosis, spinal cord injury, motor nerve injury or peripheral nerve traumatic damage.

9. (canceled)

10. (canceled)

11. (canceled)

12. (canceled)

13. (canceled)

14. (canceled)

15. (canceled)

16. (canceled)

17. A method for treating a mammal suffering from damage to the central or peripheral nerve system which comprises administrating to the mammal a therapeutically effective amount of a neural precursor cell derived from subcutaneous tissue.

18. The method of claim 17, wherein the neural precursor cell is a cell to form a neurosphere and to express nestin and/or SOX10.

19. The method of claim 18, wherein the neural precursor cell is a cell obtained from a sphere which is formed by culturing a subcutaneous tissue cell in a medium comprising N2 supplement, bFGF and EGF.

20. A method for treating a mammal suffering from damage to the central or peripheral nerve system which comprises administrating to the mammal a therapeutically effective amount of a neuron which is obtained by differentiating a neural precursor cell derived from subcutaneous tissue.

21. The method of claim 20, wherein the neuron is a cell which is obtained by differentiating the neural precursor cell in a neurobasal medium comprising NT3.

22. A method for treating a mammal suffering from damage to the central or peripheral nerve system which comprises administrating to the mammal a therapeutically effective amount of an oligodendrocyte or schwann cell which is obtained by differentiating a neural precursor cell derived from subcutaneous tissue.

23. The method of claim 22, wherein the oligodendrocyte or schwann cell is a cell which is obtained by culturing the neural precursor cell in a medium comprising retinoic acid and by differentiating the neural precursor cell in a medium comprising serum, forskolin, bFGF, PDGF and heregulin.

24. The method of claim 17, wherein the damage to the central or peripheral nerve system is Parkinson's disease, stroke, amyotrophic lateral sclerosis, spinal cord injury, motor nerve injury or peripheral nerve traumatic damage.

25. A method of preparing a neural precursor cell from a subcutaneous tissue cell which comprises culturing the subcutaneous tissue cell in a medium comprising N2 supplement, bFGF and EGF.

26. A method for differentiating the neural precursor cell derived from subcutaneous tissue cell according to claim 25 to a neuron by culturing the neural precursor cell in a neurobasal medium comprising NT3.

27. A method for differentiating the neural precursor cell derived from subcutaneous tissue cell according to claim 25 to an oligodendrocyte or schwann cell by culturing the neural precursor cell in a medium comprising retinoic acid, and then in a medium comprising serum, forskolin, bFGF, PDGF and heregulin.
